(19) United States
(12) Patent Application Publication
(10) Pub. No.: US 2008/0077066 A1
(43) Pub. Date: Mar. 27, 2008

(54) Lewis lift AFO device

(76) Inventor: Herbert Hughes Lewis, Jacksonville, FL (US)

Correspondence Address: HERBERT H. LEWIS, 5201 ATLANTIC BLVD. #118, JACKSONVILLE, FL, 32207, US

(21) Appl. No.: 11/527,376

(22) Filed: Sep. 27, 2006

(51) Int. Cl. A61F 5/00

(52) U.S. Cl. 602/28

(57) ABSTRACT

An AFO for automatically lifting the toes about the phalanges joint to assist a person when they have a medical condition referred to as “drop foot” comprising, in combination, a support that transcends vertically between two points on the lower front leg, a primary tension member attached to the upper point with a snap swivel leads to where it is fastened to the toe of a shoe at an optimum location just above the big toenail and a secondary tension member placed over the primary member so that it aids in lifting the toes and keeping them lifted. A lower strap holding the support to the front of the leg in the vicinity of the ankle does so in a spiral manner, preventing a “choking” effect that would impede circulation. The entire device is lightweight, easy on/off, comfortable and doesn't need to go in a shoe.

### FIELD OF THE INVENTION

THIS INVENTION relates to a new and novel ankle foot orthosis (AFO), and more specifically to an improved AFO device that is fastened to and worn on the lower leg to provide an attachment point for a tension member that further attaches to the toe of a shoe, slipper, or foot covering, providing lift to the foot and, in particular, the toe portion of a foot. Contributing to this is another separate tension member that when fastened to this improved AFO device functions as a human tendon, and both of these in combination greatly assist a person walk in a more normal manner with a medical condition known as “drop foot”.

### BACKGROUND OF THE INVENTION

CONVENTIONAL ankle foot orthosis called AFOs, which stands for Ankle Foot Orthosis, are used to assist a person in walking when they have a medical condition commonly referred to as “drop foot”. The drop foot condition prevents normal walking movements because the front portion of the foot, including the toe(s), does not respond to nerve signals and is unable to be raised in a spontaneously normal manner causing the foot to “droop” down at the toe. Individuals suffering from the “drop foot” malady have lost, or are seriously restricted in, the ability to voluntarily flex and move one or more of the toes, achieve upward rotation of the entire foot, may drag the foot and have difficulty walking without tripping and falling.

When walking with only one foot affected with “drop foot”, the good foot that has taken a step now supports the entire body weight; then as the other foot starts to advance by swinging forward, if the drooped front (toe) of that affected foot “stubbs”, drags on carpet, or hits any type of obstruction however slight as it advances, the “dropped foot” will stop! At that instant the forward momentum of walking could cause an immediate, and sometimes disastrous, fall.

The “drop foot” condition may exist in either the right, or left foot, and sometimes both feet are affected. If both feet are affected walking is very difficult and the person may walk by shuffling, or dragging the feet and may also require the use of other assisting devices, such as canes or walkers.

AFO devices have been on the market for years and most of them will provide support for all, or portions of, the foot. They are constructed of various combinations of materials, such as plastic, metal, or fabrics, so that they only support the foot at a roughly ninety-degree angle to the leg to keep it from drooping down. Many of them have portions that go inside of a shoe, fit and clamp around the lower leg, and some require special shoes. Most are expensive and nearly all are extremely uncomfortable to wear, especially if they must be tightly clamped around the large calf muscle of the leg, which causes extreme pain. On ones requiring insertion into a shoe, they must be cushioned in some manner by foam, layers of padding, or stockings and when all of this is put together it has to fit inside of the shoe. In addition, the rigid nature of most AFO device construction prevents ankle flex and rotation. None of the un-powered devices have the ability to raise the toes.

Since the foot is still held in a flat-out straight position at a ninety-degree angle, or less, to the heel, the use of these devices still requires a peculiar type of gait when walking. Even though the foot no longer “droops”, being held up by the AFO device, the toes are still in a flat plane with the foot. The entire foot must be raised up in order to swing forward without catching the toe and tripping and the risk of tripping and falling is still present. This also forces the person to use an unnatural circular lift, thrust forward and set down motion with the foot when walking and each step taken causes a “slapping” sound that causes embarrassment to the patient.

Most of these devices require much time to put on. Other devices built into special shoes share many of the same restrictive requirements, could require them to be individually fitted and are much more expensive. Still further devices rely on straps that fit around the leg, but these straps tend to slip down the leg, lose their effectiveness, and if excessively tight, start to put pressure on leg muscles, restrict circulation and become uncomfortable to wear for long periods of time.

In addition to the foregoing problems created with the use of conventional AFO devices, a serious problem exists by patients needing such a device not wearing them due to the time it takes to put them on, the discomforts they cause, or that they can only tolerate them for a short time. By not wearing a needed AFO the patient is highly exposed to the dangers of tripping and falling at any time.

### SUMMARY OF THE INVENTION

WHAT IS NEEDED is an AFO device for patients with “drop foot” that will not only have the means to support the foot, but also provide means to automatically raise the toes so that a normal walking gait may be achieved without tripping.

The present invention presents a new and novel ankle foot orthosis (AFO) to provide such assistance. The embodiment consists of the combination of an assembled apparatus fastened to the lower leg of the patient with a tensioning device connected to a conventional shoe and having a secondary tensioning device connected in such a manner to act as an artificial tendon to provide automatic toe lift, as well as:

An apparatus comfortable to wear for long periods of time; a means for it to be lightweight and inexpensive; means for it to be easily and quickly put on and taken off; means for two points of attachment to the lower leg for stability; the means to automatically provide additional upward force to lift up the toes to allow ground and obstacle clearance as the foot swings forward with each step for a nearly normal walking gait without tripping and falling; the means to prevent the front of the shoe (toes) from catching on an object and causing tripping; the means to attach the device to any type of foot covering at the correct location of over the base of the big toenail to provide necessary lift for the toes; the means for it to be adaptable for wear with any type of foot coverings such as shoes and slippers; the means to provide a method to prevent the toe lifting segment from putting pressure on the sides of the toes when the device is to be worn with soft foot coverings; a means to outfit multiple foot coverings with inexpensive, permanent toe lifting attachments, allowing instant and interchangeable use of different foot coverings; and the means whereby use of the apparatus by patients requiring an AFO device to be used on both feet will benefit from all the above and may not require the use of other assisting devices, all of which are a part of this invention.

### DESCRIPTION OF PRIOR ART

U.S. Pat. No. 7,077,818 discloses an ankle-foot orthosis having a structural frame formed from at least one layer of fabric impregnated with a hardened structural resin. The frame includes at least one anterior support member that extends downwardly from an upper leg engaging portion to define an anterior ankle portion which extends to a medial portion connected to a foot plate.

U.S. Pat. No. 6,926,687 discloses an ankle-foot orthosis for relief of a patient from orthopedic dysfunctions comprising, in combination, a shoe and a brace assembly. The brace assembly comprises a leg member and a pair of opposed upright members. The leg member is constructed and arranged such that portions of the inner surface of the leg member complementarily conform to portions of the rear and sides of a patient's lower leg. Each upright member of the pair of upright members is connected to the leg member and extends downwardly from the leg member to a distal end. The support brace further comprises means for resiliently connecting a portion of the support brace to the upper portion of the shoe. In use, at least a portion of the distal ends of the upright members is inserted into a portion of the heel portion of the shoe between sides of the shoe and the patient's foot.

U.S. Pat. No. 5,961,477 discloses a foot and ankle orthosis adapted for use by a patient in both resting and walking modes. The orthosis includes a generally L-shaped support member which has a leg portion positioned behind the patient's lower leg, a heel portion positioned behind the patient's heel and Achilles' tendon area, and a foot portion positioned substantially entirely against the sole of the patient's foot, when in normal operative use position. The foot portion has an upper surface and a lower surface and the heel portion connects the leg portion and the foot portion. A walking sole plate is releasably connectable to the generally L-shaped support member, to thereby permit optional use by the patient of the walking sole plate in combination with the generally L-shaped support member for facilitating ambulation by the patient. A manually operable locking mechanism is attached to the lower surface of the foot portion of the generally L-shaped support member, to thereby permit rapid, selectively; releasable connection of the walking sole plate to the generally L-shaped support member.

Other U.S. patents of possible interest relative to the present invention are U.S. Pat. Nos. 3,976,059, 4,446,856, 4,559,934, 4,651,723, 4,693,239, 4,834,078, 4,938,777, 5,088,479, 5,219,324, 5,603,692, 5,897,515, 6,299,587, 6,319,218, 6,827,696, 6,860,864, 6,908,445, 6,929,614, 6,945,947, 6,997,891, and 7,018,352.

### REFERENCE

Reference is hereby made to U.S.P.T.O. Disclosure Document 603834 dated Jul. 24, 2006 entitled “Lewis Lift AFO Device”, Herbert H. Lewis, inventor.

### DESCRIPTION OF THE DRAWINGS

A preferred embodiment of the invention is illustrated in the accompanying drawings in which:

FIG. 1 is a side view of the dropped foot condition requiring the present invention; and

FIG. 2 is a side view of an explanation for need of the present invention; and

FIG. 3 is a side view of a typical existing AFO; and

FIG. 4 is a side view of the present invention of a type used for fitting purposes; and

FIG. 5 illustrates a shoe attachment to a tension member of the present invention; and

FIG. 6 is a partial sectional view at “D-D” from FIG. 5 showing the attachment point; and

FIG. 7 is a side view of the present invention; and

FIG. 8 is a sectional view at “C-C” from FIG. 7; and

FIG. 9 is a partial side view of the spiral wrap and secondary tension member; and

FIG. 10 is a sectional view at “A-A” from FIG. 4 of the present invention; and

FIG. 11 is a sectional view at “B-B” from FIG. 7 of the present invention; and

FIG. 12 is a side view showing the secondary position of the lower leg strap; and

FIG. 13 is a graphic of the assisted walking gait, all in accordance with the invention.

### DESCRIPTION OF THE PREFERRED EMBODIMENT

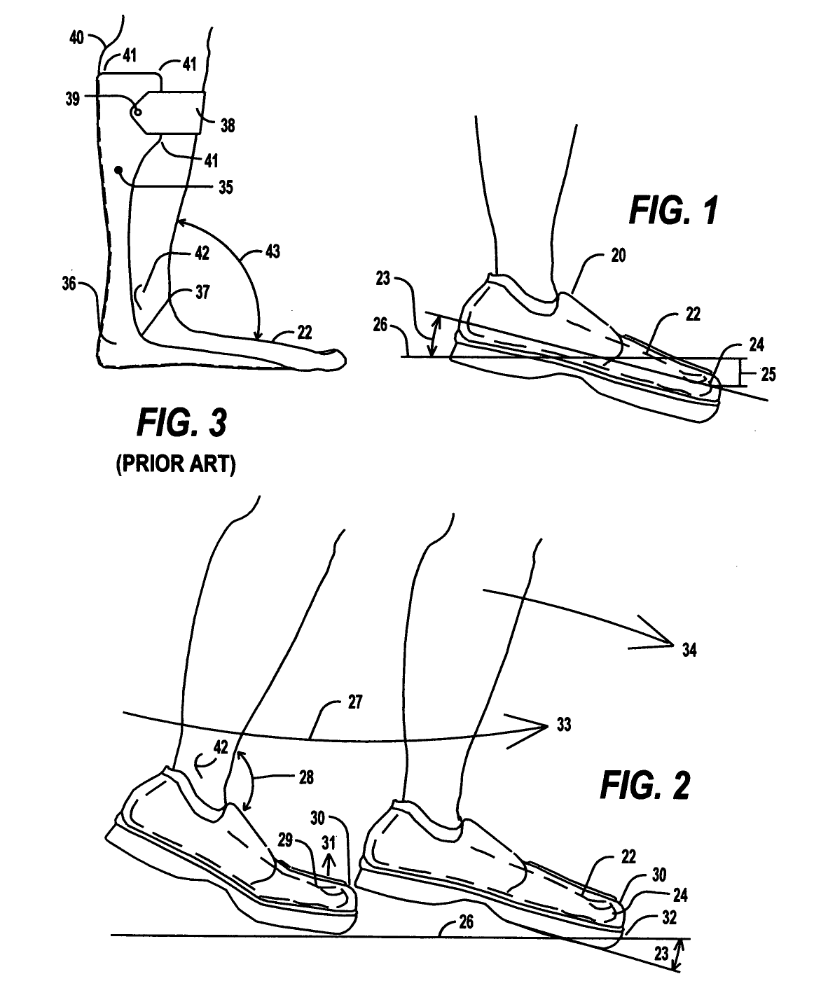

Referring more particularly to the drawings, the Numeral 1 in FIG. 1 designates an overall Side View of a shoe 20 (no other foot covering 21 shown) encasing a drop foot 22 reposing at a downward angle 23 due to a medical condition known as “drop Foot”. This is where the toes 24 of the drop foot 22 would hang below 25 a straight reference line 26 to show how the drop foot 22 in a shoe 20 would unavoidably droop down due to the problems associated with “drop foot”.

Referring now to FIG. 2: The walking swing 27 would normally flex 28 a normal foot 29 about the ankle 42 and would have the shoe toe 30 bent slightly upward 31 to clear the straight reference line 26, but a drop foot 22 having toes 24 that droop at a downward angle 23 would stub 32 the shoe toe 30 and stop the forward movement 33 of the walking swing 27, resulting in an immediate fall 34.

Referring now to FIG. 3: This is a side view of a prior art plastic AFO 35 that has a foot cup 36 that holds the drop foot 22 at roughly a ninety-degree angle 37. It is held in place by a strap 38 fastened to the plastic AFO 35 at a point 39. The placement of this device binds the calf muscle 40 at many points of contact 41 and causes much pain. The drop foot 22 is rigidly encased in the plastic AFO 35 and the ankle 42 cannot flex 43. All of this is enclosed in stockings (not shown) and thrust inside of a shoe (not shown). The inconvenience of this design causes pain and problems not found in the present invention.

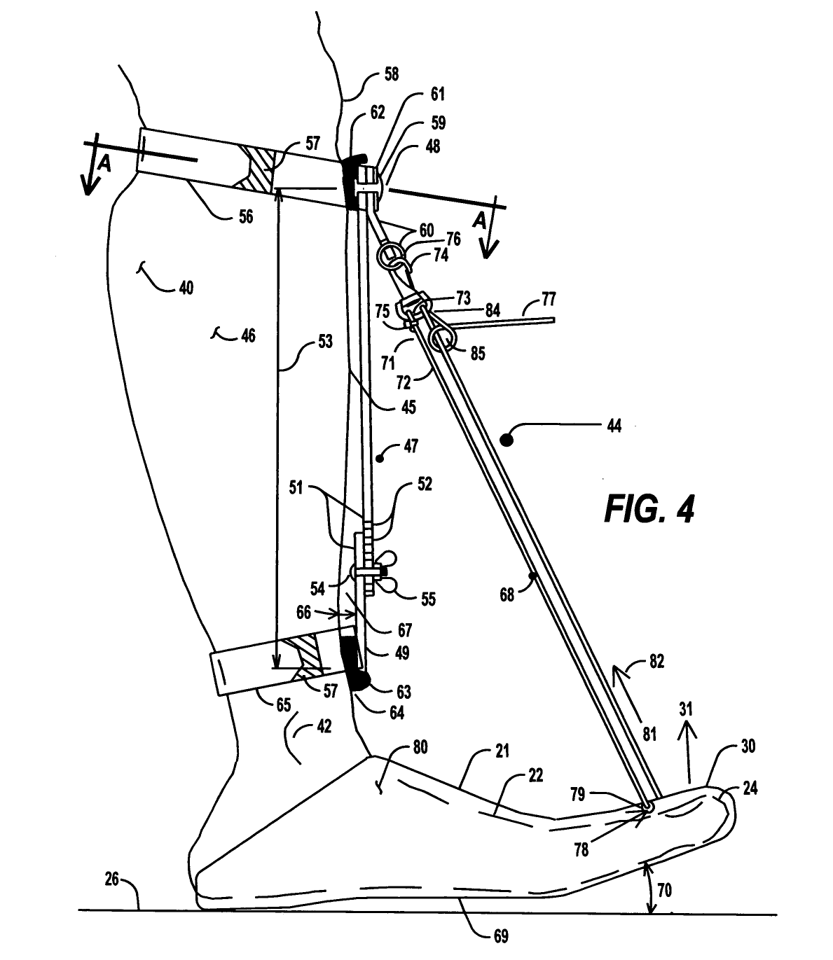

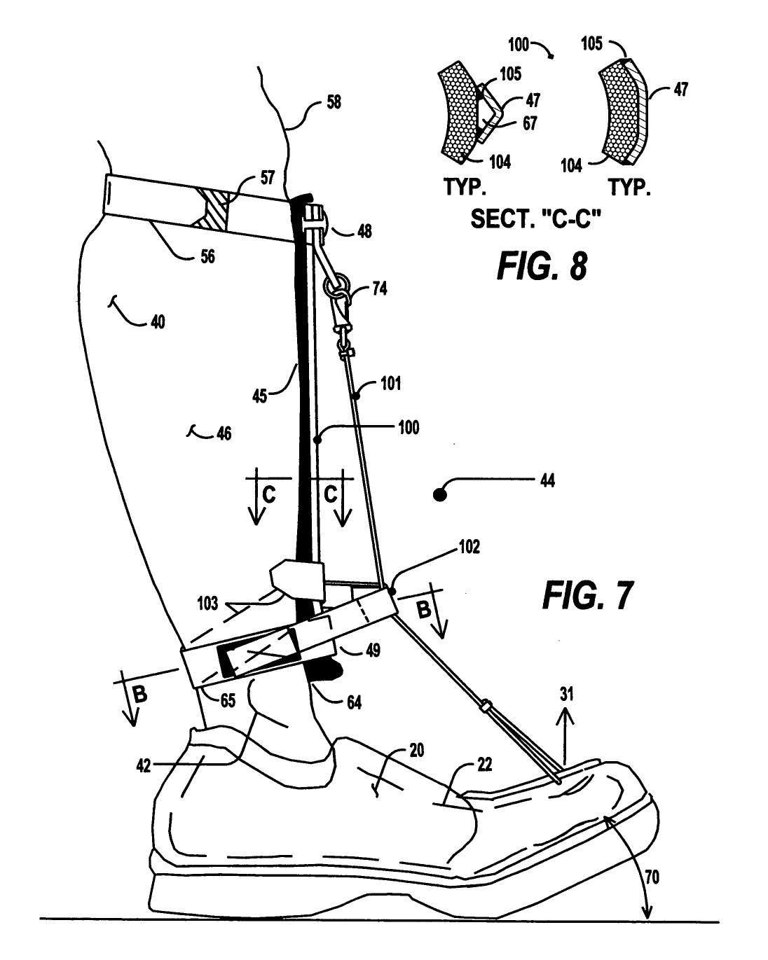

Now, FIG. 4 is a side view illustrating the present AFO invention 44 that is fastened to the front part 45 of a human leg 46 and consists of a support bar 47 with an upper attachment point 48 and a secondary attachment point 49. This support bar 47 may be of a two-piece design 51 as shown here but, this is intended for fitting purposes only. Made of various materials shaped for structural rigidity (See FIG. 8), and using adjustment holes 52 to change the length 53, a conventional machine screw 54 and wing-nut 55 holds the two-piece design 51 in place while measurements are taken. The upper attachment point 48 includes an upper strap 56 made of a conventional soft lined strapping material fitted above the calf muscle 40 and just below the knee 58 and fastened to the leg 46 with Velcro™ press together connectors 57. The upper strap 56 is connected to the support bar 47 at the upper attachment point 48 by conventional means such as a rivet 59 along with a ring assembly 60 and washers 61. The front part 45 of the leg 46 is separated and cushioned from the support bar 47 by a soft foam pad 62. A similar soft foam pad 63 at the secondary attachment point 49 cushions the front part 45 of the leg 46 at the ankle 42 and, along with a lower strap 65 made from a conventional soft lined strapping material secured to the leg 46 with Velcro™ press together connectors 57, forms a vital support point 64 for the support bar 47. This arrangement allows the support bar 47 to be held away 66 from the front 45 of the leg 46 by the soft foam pad 62 and 63 leaving an empty space 67 that prevents contact. Measurement of the length 53 of the two-piece design 51 on the present AFO invention 44, when fitted to a patient, allows customization of the device as shown in FIG. 7. Most components shown here also apply to FIG. 7 with some modification.

A further part of the AFO invention 44 shown here is a fitting version of a tension member assembly 68 that will support 69 a drop foot 22, as well as raise 70 the toes 24 above a straight reference line 26. The top end 71 of the fitting chord 72 is permanently fastened 73 to a snap 74 with a tie wrap 75. The snap 74 attaches to the ring 76 of the ring assembly 60. The lower end 77 of the fitting chord 72 is passed through 78 eyelets 79 of a fitting shoe 80 thence back up 81 with enough tension 82 applied to the fitting chord 72 to support 69 a drop foot 22 and replicate a shoe toe 30 or other foot covering 21 bent slightly upward 31. The lower end 77 of the fitting chord 72 is then temporally secured 84 with a knot 85, all for purposes of customizing the present invention by fitting to a patient.

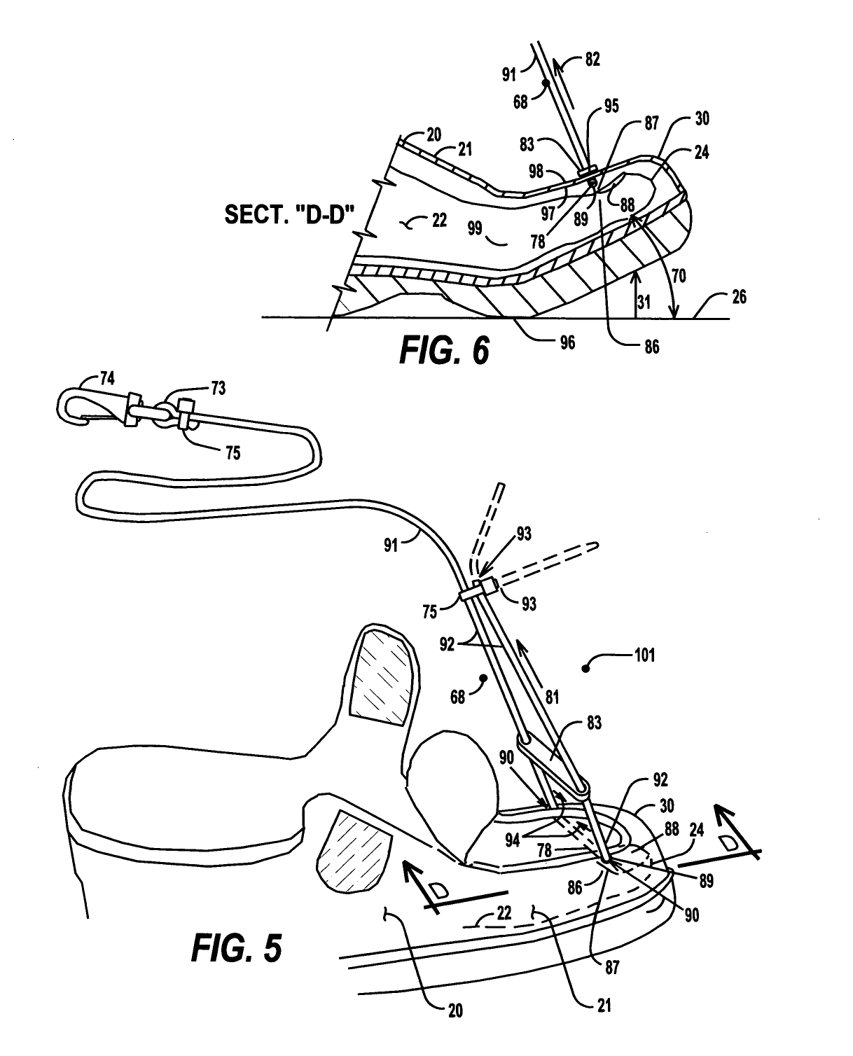

Now, FIG. 5 is an illustration showing a typical shoe 20 that could be any type of foot covering 21 with a tension member assembly 68 attached to the shoe toe 30 at the optimum location 86 directly above the base 87 of the big toenail 88 of the toes 24 on a drop foot 22. A hole 89 in each side 90 of the foot covering 21 allows the tension member 91 of the tension member assembly 68 to be passed through 78 the holes 89 and back up 81 to a wire wrap 75 that, when tightened, secures the two-lines 92 of the tension member 91 together. Excess materials 93 are trimmed off. To keep the two lines 92 of the tension member 91 from exerting side pressure 94 on the toes 24 when a soft foot covering 21 is used, a spreader bar 83 is used. To complete the tension member assembly 68 the tension member 91 is permanently fastened 73 to a snap 74 with a tie wrap 75. With all components put together and adjusted as shown in FIG. 5 and FIG. 6, the assembled combination of parts become the primary tension member and shoe assembly 101.

Accordingly, FIG. 6 is a partial sectional view “D-D” from FIG. 5 through a shoe toe 30 for showing the optimum location 86 of attachment points 95 for the tension member assembly 68 on any foot covering 21. This would be as close as possible to the location of the base 87 of the big toenail 88 on the affected drop foot 22 within the confines of the shoe 20. These attachment points 95 would provide a natural pivotal point 96 allowing the shoe toe 30 and toes 24 to be lifted upward 31 in relation to the straight reference line 26 by the amount of tension 82 applied to the tension member 91. The tension member 91 is passed through 78 holes 89 punched in the foot covering 21 just below 97 the outside surface 98. A spreader bar 83 placed on the tension member assembly 68 and pulled down to the outside surface 98 prevents the two lines 92 (not shown) of the tension member assembly 68 from putting undue pressure on the toes. The important attribute to raise the toes 70 is achieved by placing the attachment points 95 over the big toenail 88 and using the natural pivotal point 96 of the drop foot 22 at the phalanges joint 99 where the rows of toes start will become more significant as the description continues.

FIG. 7 is a side view of the present AFO invention 44 in its entirety, consisting of a primary tension member and shoe assembly 101 with a secondary tension member assembly 102, fastened in combination to a support bar assembly 100 on a human leg 46.

The support bar assembly 100 containing the upper attachment point 48 is easily fastened to the front part 45 of the leg 46 with an upper strap 56 and Velcro™ press together connectors 57 above the calf muscle 40 and just below the knee 58. Similarly, a support point 64 is established at the secondary attachment point 49 with a lower strap 65 attached to the human leg 46 in a spiral 103 configuration just above the ankle 42, refer to Section “B-B”.

A drop foot 22 is placed into a shoe 20 of the primary tension member and shoe assembly 101 and quickly connected to the support bar assembly 100 with a snap 74.

The secondary tension member assembly 102 is fastened to the support bar assembly 100 over the primary tension member and shoe assembly 101 with Velcro™ press together connectors and the orthosis has been easily made ready to support the dropped foot and raise toes 70 by providing necessary upward 31 lift.

Then, FIG. 8 is a cross section “C-C” showing how the support bar assembly 100 may be made structurally strong using various shaped support bar 47 configurations without limitation. Foam rubber padding 104 is secured by using glue 105 as required and open space 67 is not a necessity.

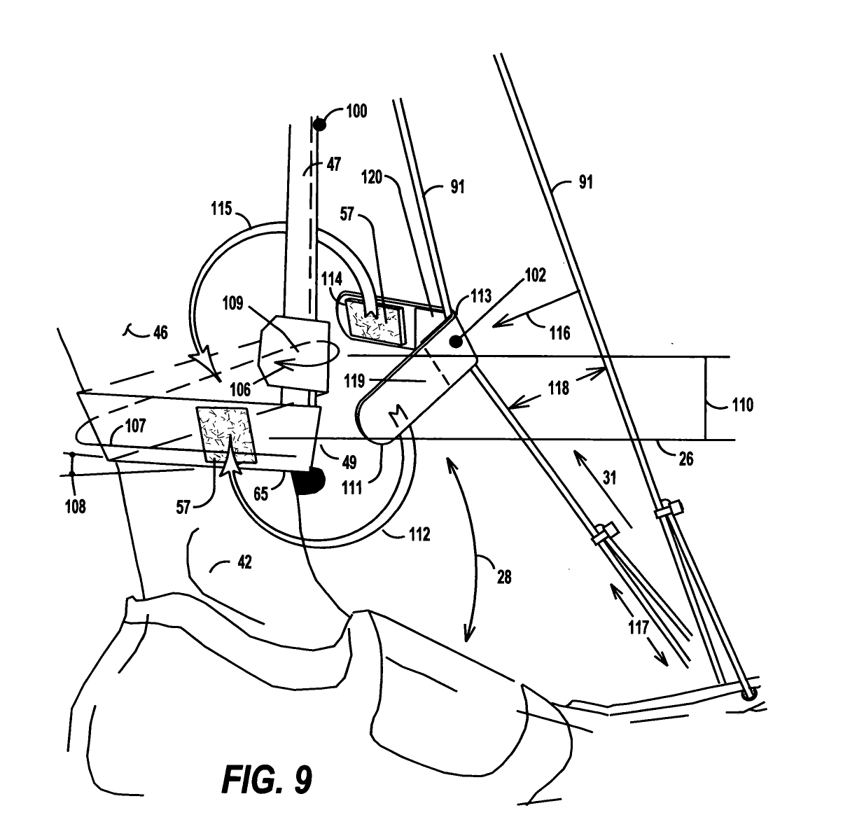

FIG. 9 shows the secondary connection of the support bar assembly 100 to the human leg 46 and how the lower strap 65 that is fastened to the support bar 47 at the secondary attachment point 49 wraps around 106 the human leg 46 in spiral 107 configurations forming an angle 108 to keep from constricting the leg. Velcro™ fasteners (not shown) attached to the lower strap end and the support bar at point 109 secure the device at some distance 110 above the straight reference line 26. Then, the end 111 of the secondary tension member assembly 102 is first attached 112 to the lower strap 65, then placed over 113 the tension member 91 and the other end 114 secondly attached 115 to the opposite side of the lower strap 65 with Velcro™ fasteners 57 to draw back 116 the tension member 91 adding/decreasing 117 tension as the ankle 42 is allowed to flex 28. The in/out 118 movement of the secondary tension member assembly 102 is imparted by elastic strips 119 and with a wear strip 120 added to increase service life. The secondary tension member assembly 102 thus acts as an artificial tendon to provide upward 31 lift.

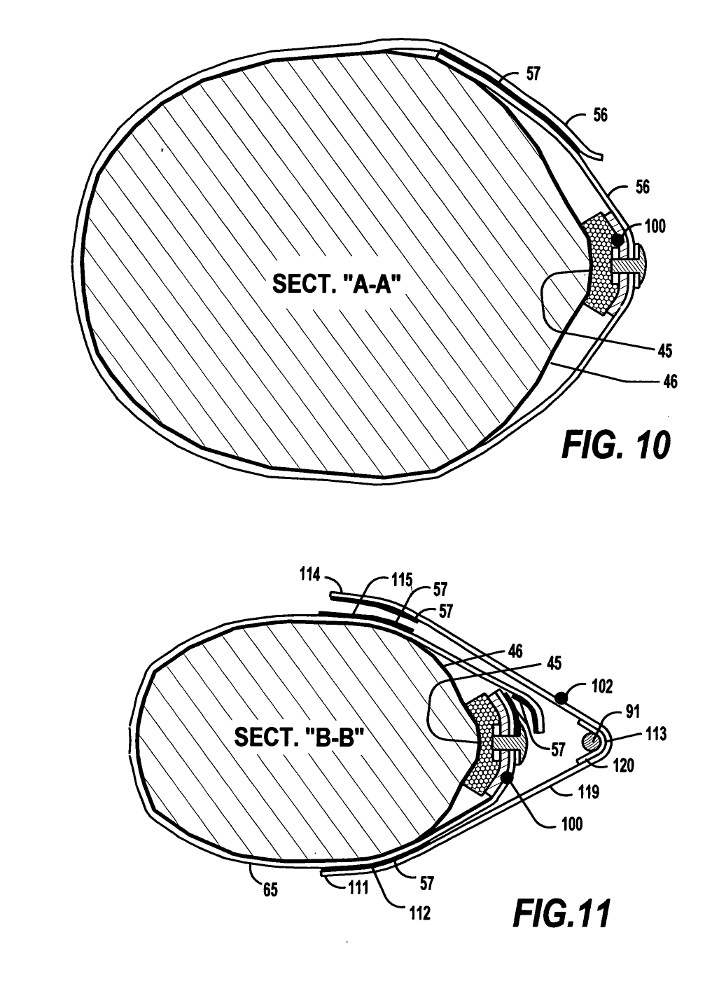

Now, FIG. 10 is a Cross Section “A-A” of the support bar assembly 100 attached to the front part 45 of a human leg 46 with the upper strap 56 wrapped around and secured with Velcro™ press together connectors 57. FIG. 11 is a Cross Section “B-B” of the support bar assembly 100 attached to the front part 45 of a human leg 46 with the lower strap 65 wrapped around and secured with Velcro™ press together connectors 57. The end 111 of the secondary tension member assembly 102 comprised of elastic strips 119 and a wear strip 120 is first attached 112 to the lower strap 65 then placed over 113 the tension member 91 and the other end 114 attached 115 with Velcro™ fasteners 57.

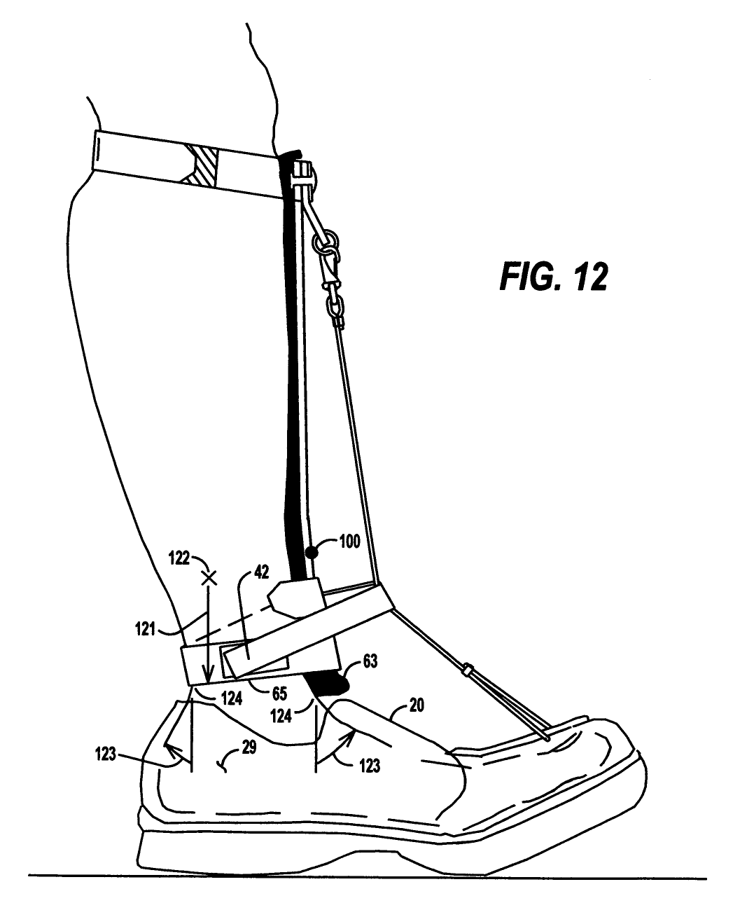

FIG. 12 is a side view of the support bar assembly 100 that has been extended 121 from the preferred location 122 so that the lower strap 65 along with the soft foam pad 63 will rest at ankle 42 level where a normal foot 29 widens 123 to provide natural stop points 124 just above the shoe 20 for a patient requiring a lower placement of the present AFO invention.

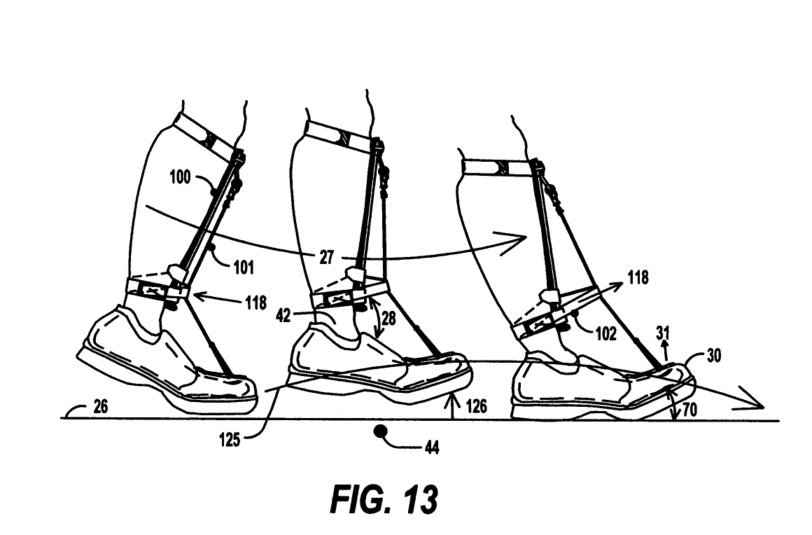

FIG. 13 illustrates the natural walking gait 125 achieved with use of the present AFO invention 44. From a reference line 26, the drop foot encased in a shoe, or other foot covering, is being supported with the toes raised up 70 to provide ground clearance 126, and with the ability of the ankle 42 to flex 28, a normal walking swing 27 may be used, all with the combined use of a support bar assembly 100, a primary tension member assembly 101 to provide the necessary upward 31 lift, and a secondary tension member assembly 102 to act as an artificial tendon providing varying in/out 118 tensioning to further provide upward lift 31 of the shoe toe 30.

Other objects, features and advantages of the present invention will become apparent upon considering the specifications and when taken in conjunction with the accompanying drawings. While a preferred embodiment of this invention has been specifically shown and described, this was for the purposes of illustration only, and not for the purposes of any limitation, the scope of the invention being in accordance with the following claims.

What I claim as my invention is:

1. An improved ankle foot orthosis, hereinafter referred to as an AFO, to provide orthopedic support and lifting of the toes for the dysfunctional condition of a human foot known as “dropped foot”, comprising in combination: a support bar assembly that fastens to the lower leg at a point on the ankle and transcends vertically up the front of the leg to an upper attachment point just below the knee, these two points being connected by a cushioned support bar with an open eyelet at the top for connection of; a primary tension member assembly one end of which attaches to the eyelet with a snap on/off connector and extends vertically downward with the other end fixed firmly to the front part of a shoe, or other type of foot covering, for the further application of; a secondary tension member assembly that is of elastic design to provide variable tensioning that has one end attached to the support bar assembly on one side of the patient's leg using Velcro™ connectors, thence passes over the primary tension member assembly to have the other end fasten to the opposite side of the leg to the primary tension member assembly with Velcro™ connectors.

2. The AFO as in claim 1, comprising in combination: a support bar assembly attached at two points on the lower leg to reduce bodily stress; means to fasten the device to the leg in order to lessen problems of circulation; means to keep the device in the correct vertical position on the front of the leg; means to provide cushioning for comfort; means for providing structural rigidity of the support bar; and means to provide an attachment eyelet at the upper end of the device.

3. The AFO as in claim 2, wherein a support bar transcends a vertical axis on the front of the leg providing structural rigidity for an upper support point that will be the fixation point of the primary tension member assembly.

4. The AFO as in claim 2, wherein a lower strap holding the support bar to the leg in the vicinity of the ankle does so in a spiral manner, so that the starting point of the strap on the support bar is significantly higher, or lower, as it finishes wrapping around the leg and fastens to the support bar with Velcro™ connectors, preventing a “choking” effect to the leg that would impede circulation.

5. An improved ankle foot orthosis (AFO) to provide orthopedic support and lifting of the toes for the dysfunctional condition of a human foot known as “dropped foot”, comprising in combination: a primary tension member assembly that has a tension member with one end fastened to a snap connector, the tension member then extends vertically downward to the toe part of a shoe, or other type of foot covering, and is firmly fixed to it, a shoe that then becomes a part of the primary tension member assembly, using holes punched, drilled, or otherwise pierced through the two sides of the shoe, at an optimum location just above the big toenail; a tension member is passed through these holes and made fast to itself by a clamping device, with a spreader bar inserted if there is danger of upward tensional force generated by the device putting pressure on the sides of the toes; a connection is made to the upper connection point of the support bar assembly by fastening the snap connector to an eyelet provided for this purpose the device is ready to apply support and provide lift to both the foot and toes.

6. The AFO as in claim 5, wherein a shoe, or any other type of foot covering such as a slipper, and hereinafter only referred to as shoe, may be fitted with an inexpensive primary tension member assembly that may be quickly donned and interchangeably worn by a patient as necessitated.

7. The AFO as in claim 6, wherein a soft type of shoe such as a slipper is fitted with a primary tension member, side forces on the toes may be generated by the upward force of the tension member as connected to each side of the shoe and a spreader bar would be placed on each of the tension members to hold them in a separated position, thus eliminating the problem.

8. The AFO as in claim 6, wherein a set of two holes may be punched transversely in a shoe on each side, pierced just under the top surface in the vicinity of the big toenail, to further provide a permanent attachment point for a tension member to be passed through the holes at this optimum point to provide adjustable lift to the toe of the shoe.

9. The AFO as in claim 8 for the tension member to be temporally adjustable for fitting purposes when used in conjunction with a fitting shoe and fitting model of a support bar assembly, the support bar of such made of two parts that adjust up/down to conform to and confirm a patient's leg length.

10. The AFO as in claim 8 wherein a tension member provides lift to the toes and assists with the rotation of the foot at the ankle, thus raising the toes which allows the affected foot to swing in a normal manner without tripping on objects and causing a fall.

11. The AFO as in claim 8 wherein an important attribute of this invention is achieved by placing the attachment points of the tension member on the toe of the shoe forward of a natural pivotal point of the foot where the rows of toes start, the phalanges joint, and at a point over the big toenail of the foot.

12. The AFO as in claim 11 wherein a lifting of the toes about the phalanges joint and keeping them in a lifted position adds greatly in providing a natural walking gait and allows the toes to clear objects naturally that would otherwise cause tripping.

13. An improved ankle foot orthosis (AFO) to provide orthopedic support and lifting of the toes for the dysfunctional condition of a human foot known as “dropped foot”, comprising in combination: a secondary tension member assembly that is a tension band made of elastic material with one end having half of a Velcro™ connector affixed to it; the center portion of the band being reinforced with a wear strip and the other end having a similar half of a Velcro™ connector attached, that, when both ends of the tension band are attached to corresponding Velcro™ connectors attached to each side of the support bar assembly and with the tension band looped over the tension member of the primary tension member assembly and the wear strip resting on the tension member, an in/out tensional force is produced with each step that further automatically lifts the toe of the shoe as the heel strikes the walking surface and the foot rotates with ankle movement, creating a device that functions as an artificial tendon.

14. The AFO as in claim 13 wherein an elastic tension band automatically adds lift to the toe of a shoe and functions as an artificial tendon for assistance in lifting the toe of a shoe.

15. The AFO as in claim 14 wherein an elastic tension band automatically works in conjunction with the ability of the foot to flex and, at the same time, is able to rotate about the ankle as the foot raises and alternately the heel strikes the ground, both of which actions are important in providing additional tension for assistance in lifting the toe of a shoe and creating a natural walking gait.

16. The AFO as in claim 1 and further including an ability of the device to be, in combination, lightweight, easily, and quickly attached to the leg without causing discomfort to the calf muscle with no insertion inside of a shoe required (except for wearing of the same).

17. The AFO as in claim 1 and further including the ability to assist a patient with “drop foot” affecting both feet so that a device is worn on each foot, allowing them all the benefits mentioned and allowing them to walk with a more normal gait, possibly without the use of other assisting devices.

18. Still further, the AFO as in claim 9 to provide medical practitioners with an inexpensive patient-fitting device that is easy and fast to adjust in order to specify the correct size.
